(19) United States
(12) Patent Application Publication
(10) Pub. No.: US 2017/0095208 A1
(43) Pub. Date: Apr. 6, 2017

(54) Device For Determining The Condition Of A Person's Skin

(71) Applicant: AIT AUSTRIAN INSTITUTE OF TECHNOLOGY GMBH, Wien (AT)

(72) Inventors: ANDREAS OBERLEITNER, ZILLINGDORF (AT); ROBERT LURF, GLOGGNITZ (AT); MICHAEL MEINDL, WIMPASSING (AT); MARTIN BEISTEINER, BAD SCHOENAU (AT)

(73) Assignee: AIT AUSTRIAN INSTITUTE OF TECHNOLOGY GMBH, WIEN (AT)

(21) Appl. No.: 15/126,349

(22) PCT Filed: Mar. 13, 2015
(86) PCT No.: PCT/AT2015/050064

§ 371 Date: Sep. 15, 2016

(30) Foreign Application Priority Data
Mar. 17, 2014 (AT) A50191/2014

(51) Int. Cl. A61B 5/00; A61B 5/053

(52) U.S. Cl. CPC A61B 5/6833; A61B 5/0002; A61B 5/443; A61B 5/445; A61B 5/0531; A61B 5/0537; A61B 5/0077; A61B 5/4839; A61B 2562/0214; A61B 2560/0412; A61B 2562/164

(57) ABSTRACT

A device for determining the condition of a person's skin includes a flat plaster sheet bindable to the skin and made of a flexible, non-conductive film with a recess for application of cosmetic or medical preparations to the skin from outside. Contact electrodes disposed on the skin as a sensor for the flat skin plaster sheet are connected by NFC technology with a voltage supply unit having an impedance and resistance and/or capacitance measuring unit. A passive communication unit with an antenna loop is supplied with measurement or skin resistance and/or capacitance data and is accessible by an NFC-enabled external communication device. The recess has a rectangular, square, circular, hexagonal or octagonal shape, pairs of diametrically opposed, associated electrodes in the flat plaster film sheet and opposite edges being rectilinear, parallel, disposed in vicinity of opposing edges of the recess and connected with the measurement unit.

The present invention relates to a device for determining the condition of the skin of a person by means of a plaster made of a non-conductive flexible material applied to at least one point of the skin under the same binding in which are embedded a sensor data measuring and processing unit connected to a sensor, an associated current or voltage supply unit, and a communication unit connected to an antenna.

The new device is intended firstly, therefore, to determine the moisture of the skin and its alteration over time and, secondly, the status of a skin lesion, a wound after injury or surgery, a pathological skin change, and the like, and their alteration over time by identifying and monitoring skin impedance.

It has long been known that one of the essential factors for the appearance and well-being of a person lies in the condition of their clothing-free skin, particularly of the facial skin, due to moisture.

Based on this knowledge, a large part of current research relates to aging and, in particular, efforts in the cosmetics sector where the main aim is to improve sustainability, in particular, of newly developed or new-to-be developed skin lotions, emulsions, creams and ointments.

To determine the effectiveness and effect of cosmetics and possibly medicinal preparations, cosmetic products and raw materials that are to be applied to the skin for this purpose, widespread methods are used to determine skin moisture, skin elasticity and trans-epidermal water loss (TEWL), as well as their changes within a respective predetermined observation period,

Today, various methods, such as, in particular, infrared spectroscopy, resonance frequency measurement, NMR methods, measurement of electrical resistance, impedance or capacitance of the skin, are customarily used to determine skin hydration. The sensor of a capacitance measuring device used to determine skin hydration contains at least one capacitor. When the device head is pressed against the skin, then the horny layer reaches the range of variation of the capacitor field, wherein use is made of the relatively high dielectric constant of water in the range of about 80 at 30° C. In principle, the dielectric constant of the skin is determined at its greatest when the skin is moist, and vice versa. The water content of the skin changes the capacitance value. The capacitance value is converted in the device into a digital measurement value corresponding to the respective moisture content of the skin.

It has thus been established in this respect, for example, that the lasting skin hydration values differ significantly in the case of a coenzyme Q10-containing cream, which sometimes does and sometimes does not contain a nano-structured lipid carrier (NLC). It has been found that the skin moisture increases significantly by applying any of the creams for more than seven days. It increases upon applying the NLC-containing cream over the entire study period, while, when using the NLC-free cream, at first it increases and then remains at a constant low level.

The new device is by no means limited to the observation of the effects of cosmetics on the skin, but is especially geared to monitor skin changes, whether caused by wounds, lesions, contamination with microorganisms or possible endocrine changes, by measuring the impedance of the resistor and/or the capacitance of the skin.

WO 2013/049716 A1 generally discloses a flexible device with a sensor for determining and ultimately displaying physical and/or chemical conditions or relationships prevailing on a surface, in particular a skin surface, and in its vicinity, wherein a sensor is provided for this measuring task connected to a processing unit for the data supplied by the sensor and a communication unit with an antenna, in particular intended for the dissemination of the calculated data to the outside, i.e. to a conventional communication device, such as a mobile phone, smartphone or the like.

US 2011/178375 discloses a device for determining the condition of a person's skin with an adhesive flat film sheet made of a non-conductive plastic and/or polymer-bound fabric sheet adapting to the skin topography applied at least to one fixed point of the skin surface under the same binding, and in which—connected to a sensor—are embedded the associated current or voltage supply unit, the sensor data processing unit and an antenna communication unit, with a substantially centrally-arranged window-like recess in the film, in particular passing transversely through a skin plaster flat sheet for visual observation of a specific skin site and/or on repeated application of a cosmetic and/or pharmaceutical preparation from the outside (as) directly onto a person's skin,
wherein at least two contact electrodes are arranged spaced apart as sensors on the skin side of the film, in particular of the skin plaster flat sheet, and can be applied directly to the skin,
wherein the electrodes are further connected to the same impedance and resistance and/or capacitance measurement unit via a voltage supply unit that can be supplied from outside (as) with current by means of NFC technology via conductors laid inside the film,
from which a passive communication unit may be supplied with the resistance and/or capacitance measurement data, while an antenna loop is also laid within the film of the plaster and at least guided around the recess, and along the edges of the film flat sheet,
which may be accessed directly from the outside as skin moisture and/or skin lesion or wound status data, and its alteration over time by means of an NFC-enabled communication device belonging to the group of mobile phones, smartphones, or readers.

The invention has the object of producing a practically manageable device that is inexpensive to produce and to use, that is applied to the observed skin site and remains there for some time without replacement over the longer term, and even provides visual observation of the reaction or the behavior of the skin, for example, as a result of cosmetic treatment, or the behavior of a wound after medical treatment that is repeated several times if appropriate, in order to provide differentiated clear information about the condition of the skin and that is capable of detecting even quite small changes in the skin condition.

The object of the invention is to provide a device for determining the skin condition as described for the prior art, but which differs considerably from this known device, characterized in that
the recess in the film plaster has a rectangular or square, circular, hexagonal or octagonal shape, and
in the film flat sheet comprises at least two by two, or two pairs of diametrically opposite straight lines, respectively assigned to one another, making a total of at least four electrodes, wherein the mutually opposed edges or borders extend parallel to each other, respectively in the vicinity of two mutually opposite edges of the recess, wherein the electrodes are connected to the impedance, resistance or capacitance measuring unit.

If, as is usual today, a communications receiving device, such as a mobile phone, reader or smartphone with an appropriate data-processing “app” is available for the conversion of impedance, resistance and/or capacitance measurements, for example, humidity or other characteristic skin parameter data, the accommodation for the measurement data processing and computing unit in the new film, in particular skin plaster flat sheet, provided for the determination of skin moisture, in any event, the very little space for the accommodation of hardware components, is also omitted.

At this Point and in Light of the Prior Art, the Invention Additionally Performs the Following:

The essential advantage of the device according to the invention lies, in particular, in the form of the recess passing through the film, in particular the skin plaster flat sheet. By means of this, it is possible for the first time, without having to detach the entire film, in particular the skin plaster flat sheet, repeatedly each time from the skin, wherein, for example, when a cosmetic to be tested is absorbed into the skin and then a further dose of cosmetic is applied, a new portion of the cosmetic may be applied directly from the outside to the exposed skin or skin wound area that is thus accessible through the recess from the outside and at least 2 pairs of mutually associated electrodes are arranged to enable this.

By means of the “permanent plaster” having a recess with the new arrangement of the electrode pairs, it is possible to observe and monitor progress, for example, of wound healing, possibly involving periodically repeated application of a medical ointment, a wound powder or the like, wherein despite avoiding the hitherto necessary change of the plaster, a visual inspection, for example of the healing process, is made possible. Examples of such “wounds” are those resulting from fire, cuts, chronic wounds, such as diabetic foot, pressure ulcers, ulcers, lupus or the like.

Thus the film, in particular a skin plaster flat sheet, no longer needs to be replaced and a new one used each time the application of a cosmetic or wound healing agent is required, and this thus fully ensures that the new application of the cosmetic is effected precisely at the site and in the area of the skin where a cosmetic or pharmaceutical preparation e.g. a cream or ointment was previously applied, usually several times.

Furthermore, the measuring electrodes are always exactly at the same distance with respect to one another at one and the same place for each successive following measurement, wherein a high degree of consistency in the measurement setup may be achieved. This is highly relevant, since it is known that impedance is strongly dependent on the distance between the electrodes, as well as the penetration depth of the measuring current and thus the measured tissue volume. In the case of manual bonding of adhesive electrodes whose positions have to be remembered exactly, or better, directly “drawn” on the respective part of the body, the risk of variations, such as due to different positions, different electrode properties as a result of type, aging or the like, varying pressure, etc. at each replacement is very high.

As a result of the features of the new claim 1 of the present invention, the multiaxial “symmetrical” arrangement and configuration of multiple electrodes with straight parallel edges facing each other, offers the dermatologist and their case histories an advantageous new effect, which when compared to previously known plasters, offers larger, very homogenous, parallel and intersecting field lines of an electric field, providing averaging of the individual skin or skin wound condition values occurring point-wise; furthermore, the main advantage is that any temporal change in the topography of the state of the area of the skin being observed may be recorded and appropriately responded to in each case.

The inventively provided arrangement and configuration of the electrodes of the skin condition determination plasters, is absolutely not apparent from the prior art and is not even remotely considered there.

Details of Other Advantages of the Invention are Given Below:
significant facilitation of use for wound assessment.
one does not need to always detach the plaster and, therefore, the area of the wound is not irritated.
the wound may optionally be left open (air drying) or also covered by means of, for example, transparent film, fabric or the like,
the wound may be inspected through the recess at any time or treated with creams, powders or the like,
nevertheless, the measurement setup, when compared with a previously usual manual sticking of individual adhesive electrodes, is automatically constant.

With respect to the areas of application for the new device, on the one hand there is scientific research and development, in particular in the fields of cosmetics and medicine, and, on the other hand, to a much larger extent, practice tests on persons and, in particular, their treatment, i.e. in particular in the context of wound assessments.

Thus, the invention ensures much more secure measurement results, in particular, wherein the parallel course of the field lines between the electrodes of each electrode pair is ensured.

According to an advantageous embodiment of the invention, it is provided that the distance between the diametrically-opposite edges of the recess and the diametrically opposite edges of the electrodes adjacent to the recess lies between 2 and 10 mm and/or the ratio of the opening width of the recess between the edges close to the recess and the spacing of the mutually facing, mutually parallel edges of the contact electrodes is at least 3 to 10, wherein the length of the edges of the individual electrodes close to the edge of the recess amounts to 75 to 125% of the length extension of the sides of the straight edges of the recess to the individual electrodes or to the chords of the circle of a circular recess corresponding to the number of electrodes.

It should be emphasized at this point that the above-mentioned shapes of the recess are essential.

Advantageous Arrangements of the Electrodes or Pairs of Electrodes May be as Follows:
multiple pairs (two-wire method) or quadruple (four-wire method) in parallel for the purpose of redundancy, or to determine one-dimensional discretization,
two or more pairs (two-wire method) or quadruple (four-wire method) orthogonal to each other (useful for the detection and assessment of anisotropy)
more, preferably 8 pairs or quadruples of octagonal or circular electrodes arranged around a circular recess to form two-dimensional maps using mathematical methods (e.g. linear rear projection). Concrete application: for example, monitoring the development of wound sizes.

As for the energization of the electrodes, the following variants and methods may, for example, be considered for this purpose; direct and alternating current, constant current and constant voltage method, single frequency, multifrequency or sweep ‘(typically 0 to 2 MHz).

Furthermore, it is advantageous if at least one of the pairs of electrodes is each formed with two sub-electrodes, one for the power input and one for the voltage measurement per electrode.

To avoid influences and disturbances of the measurement results by outside influences as far as possible, it is favorably provided that a planar shield unit is arranged in the form of a fine mesh or a film of an electrically-conductive material within a layer between the upper surfaces of the electrodes, lie the voltage supply unit, the measuring unit, the communication unit and their conductive connections, on the one hand, and, as close to the outer edge or the recess in the space between their edge and the edge of the electrodes close to the recess of the film flat sheet, a ring-like self-contained NFC antenna on the other hand.

In order to obtain reproducible results, it is advantageous to ensure good surface contact between the measuring electrode and the skin of each person to be tested, wherein the undersides of the electrodes directly-applied to the skin are provided, or may be provided, with a contact enhancement layer.

In particular, for reasons of space, it is advantageous if the impedance, resistance or capacitance measuring unit, an optional central data processing unit, and the communication unit are arranged within a common chip.

As such, the antenna may, in particular, be placed next to the measuring unit in the plaster, which in fact increases the area of the plaster, but does not interfere with it in practice.

Furthermore, it should be mentioned that the power supply of the measuring units and the other components may be affected by means of induction or magnetic coupling through the communication device from the outside or, if appropriate, by using a mini battery integrated in the film, in particular in the skin plaster flat sheet.

Finally, in particular, to avoid as far as possible irritation of often sensitive skin by the new plaster-like device, it is provided that the film, in particular the skin plaster flat sheet is coated with a skin friendly self-adhesive material on the under or skin side.

The Invention is Explained in Detail with Reference to the Drawings:

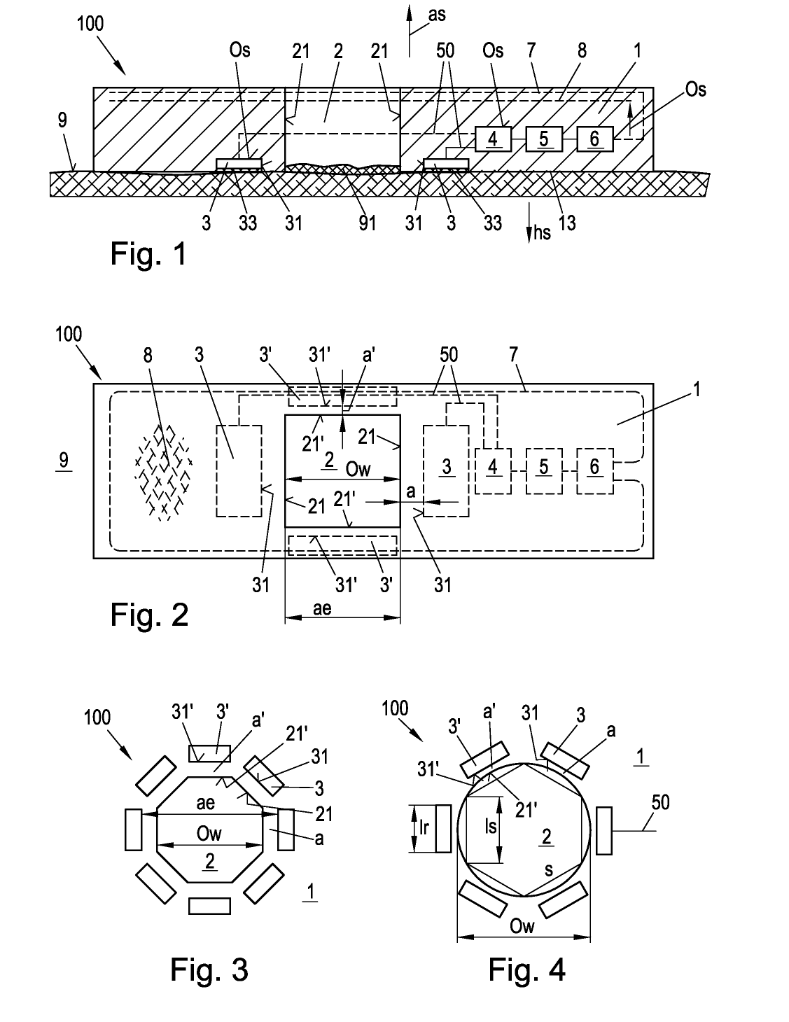

FIG. 1 and FIG. 2 show sectional and plan views of an invention typical plaster-like device 100, for example, for skin moisture determination, wherein the film, especially skin plaster flat sheet 1 is applied flat by means of an adhesive or (self-) adhesive layer 13, for example, on an area of the skin 9 to be examined of a person, while FIGS. 3 and 4 show various other possible forms of electrode arrangements and recesses.

It may be clearly seen from FIG. 1 and FIG. 2 that the film formed with a non-conductive plastic or fabric sheet, in particular a skin plaster flat sheet 1 through which a recess 2, almost square here, fully passes through the central region, wherein its respective mutually parallel opposite edges 21, 21′, along whose respective mutually parallel opposite edges 31 and 31′ the mutually associated measuring electrodes 3 and 3′ extend, and which measuring electrodes have a contact enhancement layer 33 on the underside or on the skin side (hs), to make electrical contact with the skin 9 directly or via the contact enhancement layer 33.

A layer of, for example, a cosmetic 91 containing moisture that is to be tested over the longer term is applied to the skin 9, i.e. the area of skin at the base of the recess 2, which may be observed through the recess 2 from the outside (as).

Similarly, the recess 2 may be arranged over a wound of the skin 9, wherein, here also, a medicinal ointment, a powder or the like may be periodically applied to the wound.

The electrodes 3, 3′ are connected with an impedance, resistance, or capacitance-measuring unit 4, a measurement data processing and computing unit 5 and a communication unit 6 via conductors 50 to which in turn is connected a conductor loop or antenna 7 that is guided around the recess 2 for (radio) connection with an external receiver (not shown), such as a mobile phone, reader or smartphone, by means of which the acute skin moisture or skin condition values are provided, wherein a shield grid 8 or the like may be provided between the components 3 to 6 or their upper side (Os) and the conductor loop 7.

FIGS. 3 and 4 show different forms of the recess 2 of the new plaster-like device 100 and possible inventive arrangements of the free electrodes 3, 3′ around the edges 21, 21′ of the recess 2 and embedded in the sheet material.

FIG. 3 shows an octagonal shape having a recess 2, wherein the rectangular electrodes 3, 3′ here are arranged substantially parallel to the here equally long octagonal sides (s).

The electrodes 3, 3′ here have a length extension (lr) of their edges or borders 31, 31′ close to the recess of about 100% of the length (ls) of the individual octagonal sides (s).

FIG. 4 shows a recess 2 in circular form with a hexagon drawn therein with a hexagonal section chord (s) at a distance a, a′ from the edge 21, 21′ or recess with six electrodes 3, 3′. Their extension (lr) here corresponds to 75% of the length (l) of the individual section chord (s) inscribed in the circle.

1-11. (canceled)

12. A device for determining the condition of a person's skin, the device comprising:
an adhesive flat film sheet formed of at least one of a non-conductive plastic or a polymer-bound fabric to be adapted to a topography of the skin, said flat film sheet having a skin side to be applied at least to one fixed point of the surface of the skin by a binding and having edges;
said flat film sheet having a centrally-disposed recess passing transversely through said flat film sheet for at least one of visual observation of a particular area of the skin or for a repeated application of a cosmetic or pharmaceutical preparation directly on the skin of the person from outside the device, said recess having a rectangular, square, circular, hexagonal or octagonal shape with mutually opposite edges;
at least four electrodes being associated with one area, being disposed as a sensor on said skin side of said flat film sheet and being directly applied to the skin said electrodes being disposed in at least two diametrically opposed, mutually associated and spaced-apart pairs and said electrodes having mutually parallel and mutually opposed edges or borders being straight lines each disposed in a vicinity of a respective one of two of said mutually opposite edges of said recess;
conductors laid within said flat film sheet and connected to said contact electrodes;
a current or voltage supply unit embedded in said flat film sheet, connected to said conductors and supplied with current from outside the device by NFC technology;
an impedance, resistance or capacitance measurement unit embedded in said flat film sheet and connected to said supply unit;
a passive communication unit being embedded in said flat film sheet and supplied with impedance, resistance or capacitance measurement data from said measurement unit; and
an antenna loop connected to said passive communication unit, laid within said flat film sheet and at least guided at least around said recess and along edges of said flat film sheet to permit the measurement data and its alteration over time to be accessed directly from outside the device as at least one of skin moisture or skin lesion or wound status data by an NFC-enabled communication device selected from the group consisting of mobile phones, smart phones or readers.

13. The skin condition determining device according to claim 12, wherein:
said opposite edges of said recess are diametrically opposite;
said opposite edges of said electrodes are diametrically opposite;
said diametrically-opposite edges of said recess and said diametrically-opposite edges of said electrodes adjacent said recess are spaced apart by a distance of between 2 and 10 mm and/or an opening width between said diametrically-opposite edges of said recess and a spacing between said mutually opposed, mutually parallel edges of said contact electrodes are in a ratio of at least 3 to 10;
said sides of said straight edges of said recess define a length extension to said individual electrodes or to chords of a circle of a circular recess corresponding to a number of said electrodes; and
said edges of said individual electrodes in a vicinity of said edge of said recess have a length being 75 to 125% of said length extension.

14. The skin condition determining device according to claim 12, wherein at least one of said pairs of electrodes includes one sub-electrode for power input and one sub-electrode for voltage measurement.

15. The skin condition determining device according to claim 12, wherein:
said electrodes have upper sides;
a flat planar shield unit formed of a fine mesh or a film of an electrically-conductive material is disposed between said upper sides of said electrodes;
said voltage supply unit, said measuring unit, said communication unit and said conductors are disposed in said flat planar shield unit; and
said antenna loop is an annular, self-contained NFC antenna disposed in a vicinity of said outer edge of said flat film sheet or in a vicinity of said recess in a space between said edge of said recess and said edge of said electrodes.

16. The skin condition determining device according to claim 12, wherein said electrodes have undersides directly applied to the skin, and a contact enhancement layer to be applied to said undersides.

17. The skin condition determining device according to claim 12, wherein said impedance, resistance or capacitance measuring unit and said communication unit are disposed within a common chip.

18. The skin condition determining device according to claim 12, wherein said communication unit provides an electromagnetic coupling from outside the device or said flat film sheet has an integrated mini battery, for supplying power to said electrodes and other components.

19. The skin condition determining device according to claim 12, wherein said flat film sheet is coated with a skin-friendly self-adhesive material on said skin side and said skin side is an underside.

20. The skin condition determining device according to claim 19, wherein said flat film sheet is a flat skin plaster sheet.
